(19) United States
(12) Patent Application Publication
(10) Pub. No.: US 2024/0066267 A1
(43) Pub. Date: Feb. 29, 2024

(54) Fluid Transfer Device with Deflection Biasing Catheter Support

(71) Applicant: Becton, Dickinson and Company, Franklin Lakes, NJ (US)

(72) Inventors: Yiping Ma, Layton, UT (US); Adam J. Boud, Bluffdale, UT (US); Weston F. Harding, Lehi, UT (US)

(21) Appl. No.: 17/899,099

(22) Filed: Aug. 30, 2022

(51) Int. Cl. A61M 25/01

(52) U.S. Cl. CPC A61M 25/0133; A61M 25/0113; A61M 2025/0681

(57) ABSTRACT

A fluid transfer device for advancing a catheter into an indwelling catheter includes an introducer having a proximal end portion and a distal end portion, an actuator movably coupled to the introducer and configured to move relative to the introducer to move the catheter between a first position, in which the catheter is disposed within the introducer, and a second position, in which a distal end portion of the catheter is disposed beyond the distal end portion of the introducer. The fluid transfer device also includes a catheter support movably coupled to the introducer, and positioned between the actuator and the distal end portion of the introducer, the catheter support defining a passageway therein that receives the catheter to provide support thereto. The passageway in the catheter support is configured to encourage a downward bowing of the catheter in the catheter support and/or pre-bias a downward bowing of the catheter.

### BACKGROUND OF THE INVENTION

### Field of the Invention

The present invention is directed to a fluid transfer device for introducing a catheter into an indwelling catheter of a vascular access device.

### Background of the Invention

Catheters are frequently utilized to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples.

Recent developments in the PIVC field have led to the emergence of technologies designed to facilitate fluid transfer using an in-dwelling PIVC and accompanying fluid transfer device. The main method by which these devices work is by employing an introducer for inserting a catheter, probe, tube, or other instrument through the catheter lumen of the PIVC, with the introducer being attached to a catheter adapter of the PIVC that provide for insertion of the catheter. For example, the catheter adapter may include a needleless access connector thereon by which the catheter may be introduced to provide access to the PIVC and into the patient's vasculature. A syringe and/or vacutainer may then be used to inject fluid into the patient or collect blood samples without needing to subject the patient to additional needle sticks.

The introducer of a fluid transfer device typically includes a housing, a catheter movable within the housing so as to be extendable out therefrom for advancement into the in-dwelling PIVC, and an advancement member or actuator that may be actuated by an operator relative to the housing. That is, the actuator may be moved distally by the operator to cause a corresponding movement of the catheter relative to the housing, such that the catheter may be advanced out from the distal end of the housing and into the in-dwelling PIVC. However, during deployment of the catheter of the fluid transfer device, especially when the distal end portion of the catheter reaches a curved region of the PIVC (near where the PIVC enters the skin) and is caused to curve back and forth as it passes along the vein, the catheter of the fluid transfer device is subject to a column load which can cause bending, kinking, and/or deformation of the catheter. As the catheter bends, it can move in random directions creating a sinusoidal wave, which then flattens against the sidewall of the housing of the fluid transfer device as the force increases. Additional force applied to the catheter by further movement of the actuator can then cause the catheter to double back on itself and collapse—i.e., to “buckle.”

In order to address the issue of buckling of the catheter, some introducers include a support member or catheter support that is positioned within the introducer housing at a location between the actuator and the distal end of the housing. The catheter is routed through the catheter support to reduce the length of unsupported tube, effectively increasing the resistance the flow tube can withstand before buckling. However, when the loads applied to a tube are sufficiently high, the tube could still bow. As the tube is offset vertically downward within the housing (in order to accommodate a portion of the actuator within the housing), the upper clearance is much larger than the lower clearance, and the tube tends to bow upward when a high enough load is applied thereto. When the tubing bows upwards, any additional force applied to the actuator by a user will serve more to deform and buckle the tube than push it forwards, as compared to if the tubing were to bow downwards, then the force applied to the actuator will serve more to advance the tubing than deform the tube.

Accordingly, a need exists for a fluid transfer device and introducer thereof that provides for advancement of a catheter into the PIVC while minimizing the potential for bending, kinking, or deformation of the catheter. Additionally, in the event that the catheter experiences loads sufficient to cause bowing thereof, the fluid transfer device and introducer should desirably bias the tube in a downward direction, so as to minimize the chances of buckling of the catheter.

### SUMMARY OF THE INVENTION

Provided herein is a fluid transfer device for advancing a catheter into an indwelling catheter of a vascular access device. The fluid transfer device includes an introducer having a proximal end portion and a distal end portion, the distal end portion coupleable to the vascular access device, and the introducer defining an inner volume configured to movably receive the catheter. The fluid transfer device also includes an actuator movably coupled to the introducer and configured to move relative to the introducer to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter. The fluid transfer device further includes a catheter support movably coupled to the introducer, and positioned between the actuator and the distal end portion of the introducer, the catheter support defining a passageway therein that receives the catheter to provide support to a length of the catheter that extends between the actuator and the distal end portion of the introducer. The passageway in the catheter support is configured to encourage a downward bowing of the catheter in the catheter support and/or pre-bias a downward bowing of the catheter.

In some embodiments, wherein the catheter support comprises a bracket portion and a hub portion, the hub portion including a top surface and a bottom surface, with a center axis of the hub portion extending lengthwise between a proximal end and a distal end of the hub portion.

In some embodiments, the passageway includes a proximal opening and a distal opening, and an open slotted passageway extending between the proximal and distal openings, the open slotted passageway having a top portion generally aligned with the distal opening and a bottom portion vertically below the top portion and that is open along the bottom surface of the hub portion, wherein the catheter is encouraged to bow downward within the open slotted passageway, from the top portion down toward the bottom portion.

In some embodiments, the passageway includes a proximal opening and a distal opening, and a closed slotted passageway extending between the proximal and distal openings, the closed slotted passageway having a top portion generally aligned with the distal opening and a bottom portion vertically below the top portion, with the bottom portion ending at a location vertically between the center axis and the bottom surface of the hub portion, and wherein the catheter is encouraged to bow downward within the closed slotted passageway, from the top portion down toward the bottom portion.

In some embodiments, the proximal opening, the distal opening, and the top portion are aligned with a tubing path along which the length of the catheter extends between the actuator and the distal end portion of the introducer.

In some embodiments, the proximal opening, the distal opening, and the top portion are offset vertically downward from a tubing path along which the length of the catheter extends between the actuator and the distal end portion of the introducer, thereby preloading the catheter to force a downward bowing thereof.

In some embodiments, the catheter is allowed to only bow downward at a location of the catheter support, responsive to the actuator moving along the housing to advance the catheter from the first position toward the second position.

In some embodiments, the passageway is a curved passageway, with a center region of the passageway between the proximal end and the distal end of the hub portion being offset vertically upward from a proximal opening and a distal opening of the passageway, the curved passageway pre-biasing a downward bowing of the catheter.

In some embodiments, the passageway is a sinusoidal passageway that shifts vertically as it runs between the proximal end and the distal end of the hub portion, the sinusoidal passageway pre-biasing a downward bowing of the catheter.

In some embodiments, the proximal end of the hub portion is configured to engage the actuator, such that during movement of the actuator to move the catheter from the first position to the second position, the actuator contacts the proximal end to move the catheter support with respect to the introducer.

In some embodiments, the distal end portion of the introducer comprises a lock configured to couple the introducer to the vascular access device.

Another fluid transfer device for advancing a catheter into an indwelling catheter of a vascular access device is provided that includes an introducer having a proximal end portion and a distal end portion, the distal end portion coupleable to the vascular access device, and the introducer defining an inner volume configured to movably receive the catheter. The fluid transfer device also includes an actuator movably coupled to the introducer and configured to move relative to the introducer to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter. The fluid transfer device further includes a catheter support movably coupled to the introducer, and positioned between the actuator and the distal end portion of the introducer, the catheter support including a bracket portion and a hub portion, with the hub portion defining a passageway therein that receives the catheter to provide support to a length of the catheter that extends between the actuator and the distal end portion of the introducer. The passageway includes a proximal opening and a distal opening, and a slotted passageway extending between the proximal and distal openings, the slotted passageway configured to encourage a downward bowing of the catheter within the slotted passageway.

In some embodiments, the slotted passageway is an open slotted passageway including a top portion generally aligned with the distal opening and a bottom portion vertically below the top portion, with the bottom portion ending at a location vertically between the center axis and the bottom surface of the hub portion, and wherein the catheter is encouraged to bow downward within the closed slotted passageway, from the top portion down toward the bottom portion.

In some embodiments, the slotted passageway is a closed slotted passageway including a top portion generally aligned with the distal opening, and a bottom portion vertically below the top portion, with the bottom portion ending at a location vertically between the center axis and the bottom surface of the hub portion, and wherein the catheter is encouraged to bow downward within the closed slotted passageway, from the top portion down toward the bottom portion.

In some embodiments, the proximal opening, the distal opening, and the top portion are aligned with a tubing path along which the length of the catheter extends between the actuator and the distal end portion of the introducer.

In some embodiments, wherein the proximal opening, the distal opening, and the top portion are offset vertically downward from a tubing path along which the length of the catheter extends between the actuator and the distal end portion of the introducer, thereby preloading the catheter to force a downward bowing thereof.

In some embodiments, a proximal end of the catheter support is configured to engage the actuator, such that during movement of the actuator to move the catheter from the first position to the second position, the actuator contacts the proximal end to move the catheter support with respect to the introducer.

Another fluid transfer device for advancing a catheter into an indwelling catheter of a vascular access device is provided that includes an introducer having a proximal end portion and a distal end portion, the distal end portion coupleable to the vascular access device, and the introducer defining an inner volume configured to movably receive the catheter. The fluid transfer device also includes an actuator movably coupled to the introducer and configured to move relative to the introducer to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter. The fluid transfer device further includes a catheter support movably coupled to the introducer, and positioned between the actuator and the distal end portion of the introducer, the catheter support comprising a bracket portion and a hub portion, with the hub portion defining a passageway therein that receives the catheter to provide support to a length of the catheter that extends between the actuator and the distal end portion of the introducer. The passageway comprises a non-linear passageway configured to pre-bias a downward bowing of the catheter as the catheter enters and exits the catheter support.

In some embodiments, the non-linear passageway is an arcuate passageway, with a center region of the arcuate passageway between the proximal end and the distal end of the hub portion being offset vertically upward from a proximal opening and a distal opening of the passageway, the curved passageway pre-biasing a downward bowing of the catheter.

In some embodiments, the non-linear passageway is sinusoidal passageway that shifts vertically as it runs between the proximal end and the distal end of the hub portion, the sinusoidal passageway pre-biasing a downward bowing of the catheter.

### BRIEF DESCRIPTION OF THE DRAWINGS

FIG. 1 is a perspective view of a fluid transfer device with which embodiments of the present application may incorporated;

FIG. 2 is a top view of the fluid transfer device illustrated in FIG. 1;

FIG. 3 is an exploded view of the fluid transfer device of FIG. 1;

FIG. 4 is a perspective view of a first member of an introducer housing included in the fluid transfer device of FIG. 1;

FIG. 5 is a perspective view of a second member of the introducer housing included in the fluid transfer device of FIG. 1;

FIG. 6 is a rear perspective view of the introducer housing formed by coupling the first member illustrated in FIG. 4 to the second member illustrated in FIG. 5;

FIG. 7 is a front perspective view of the introducer housing illustrated in FIG. 6;

FIG. 8 is a cross-sectional view of the introducer taken along the line 8-8 in FIG. 6;

FIG. 9 is a rear perspective view of a lock included in the fluid transfer device of FIG. 1;

FIG. 10 is an exploded perspective view a catheter, a secondary catheter, and an actuator included in the fluid transfer device of FIG. 1;

FIG. 11 is a perspective view of the actuator illustrated in FIG. 10;

FIG. 12 is a cross-sectional view of the fluid transfer device taken along the line 12-12 in FIG. 2;

FIG. 13 a rear perspective view of a catheter support included in the fluid transfer device of FIG. 1;

FIG. 14 is a front perspective view of the catheter support of FIG. 13;

FIG. 15 is a perspective view of a portion of the fluid transfer device of FIG. 1 with a second introducer housing portion removed;

FIG. 16A is a rear perspective view of a catheter support that may be included in the fluid transfer device of FIG. 1, in accordance with an embodiment of the invention:

FIG. 1613 is a front perspective view of the catheter support of FIG. 16A;

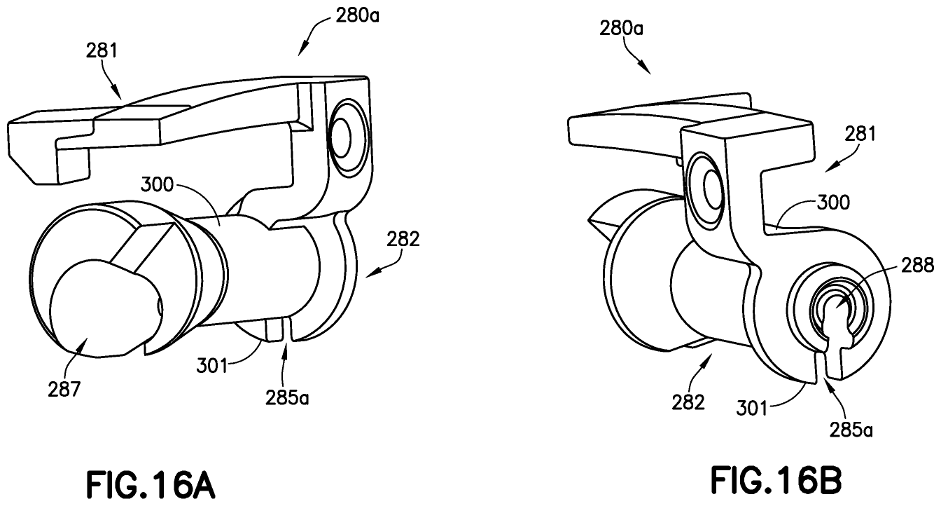

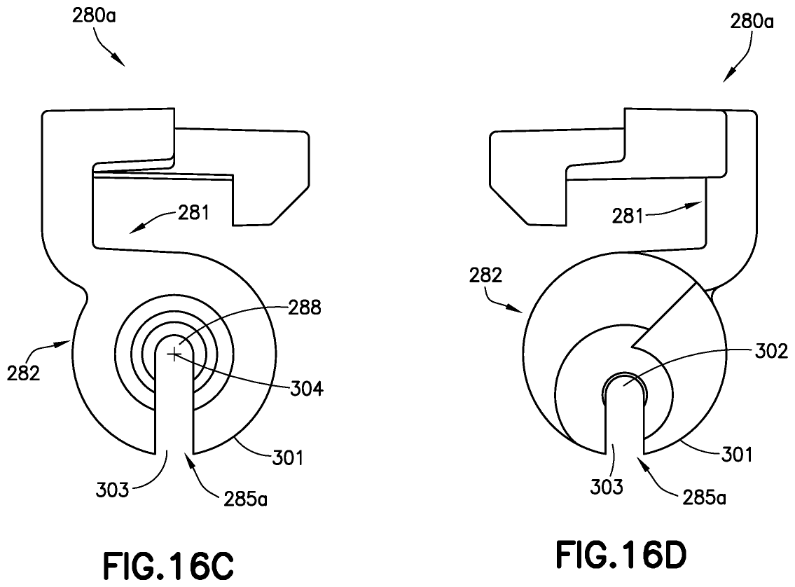

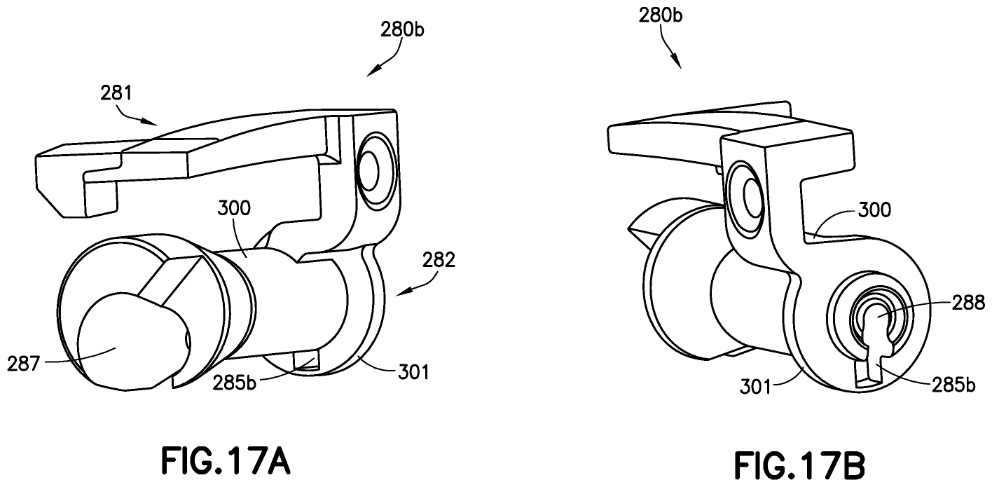

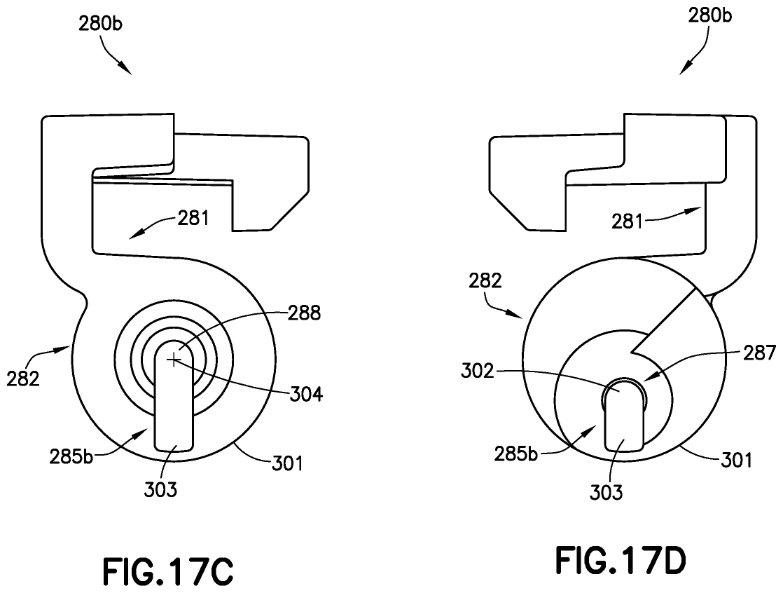

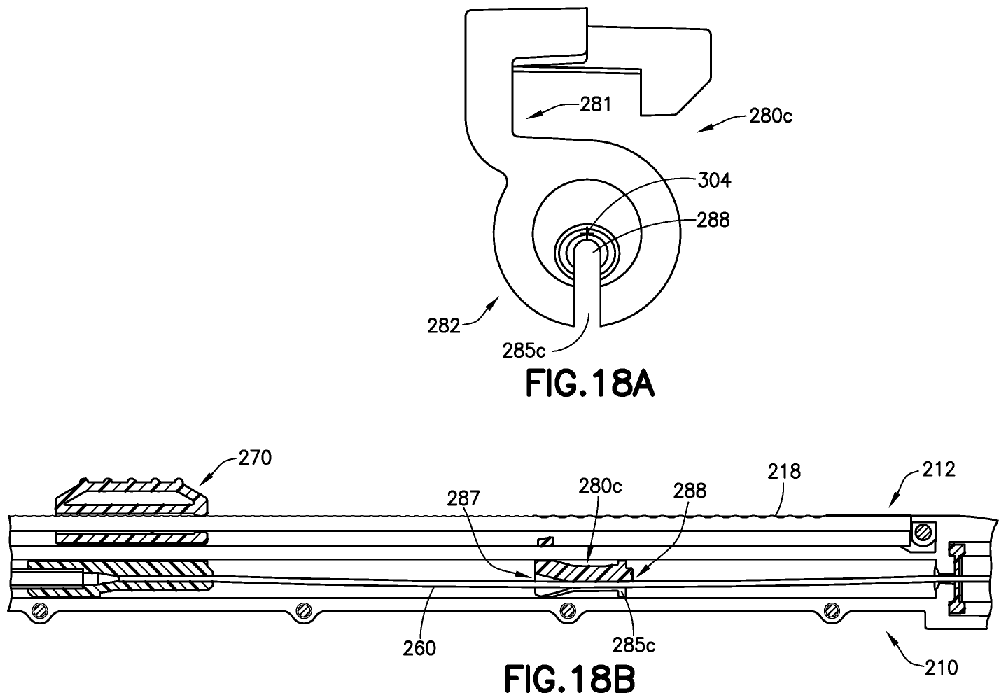

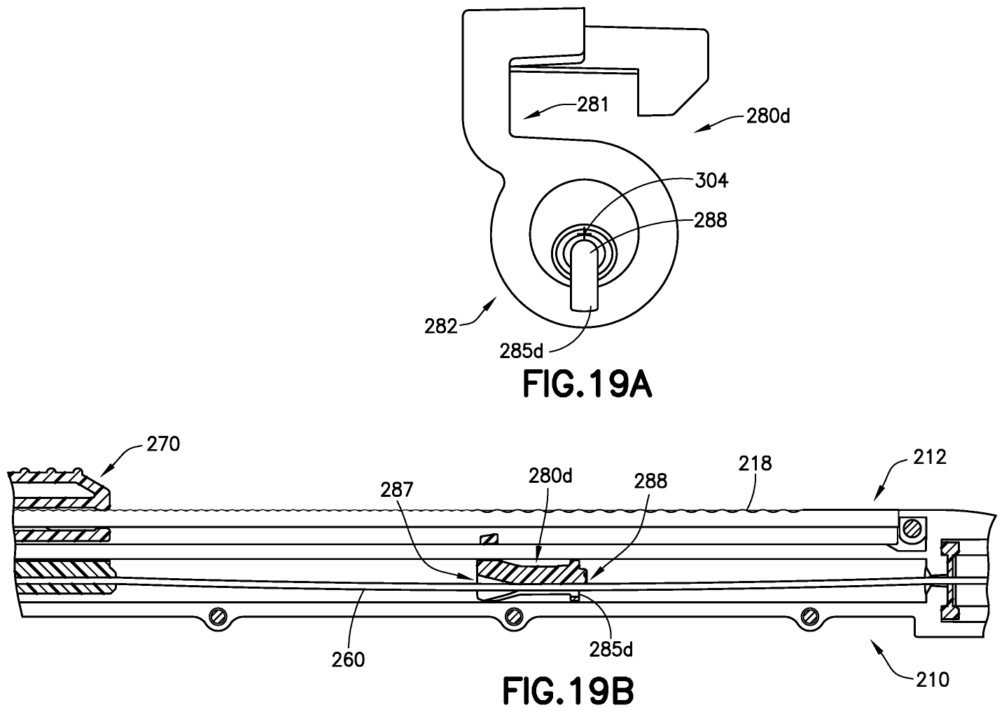

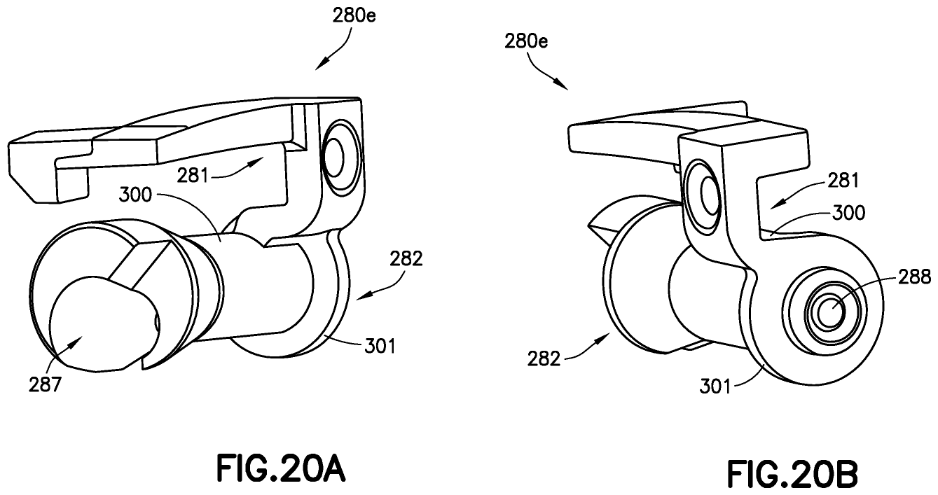

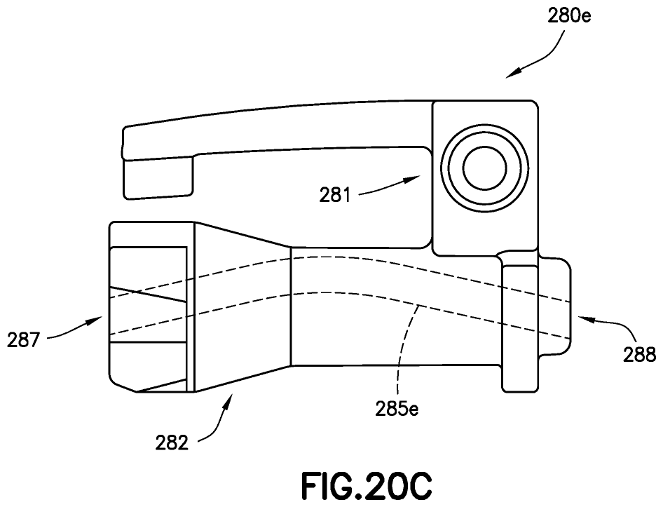

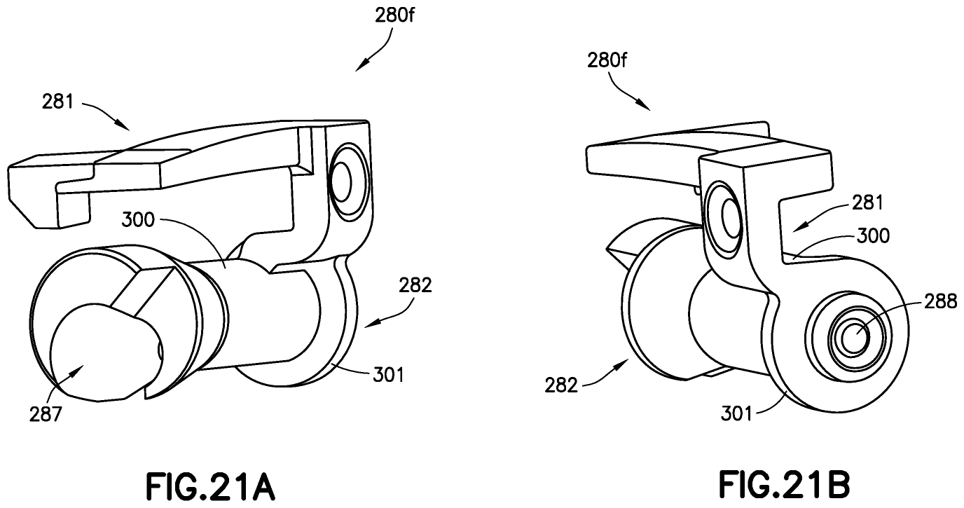

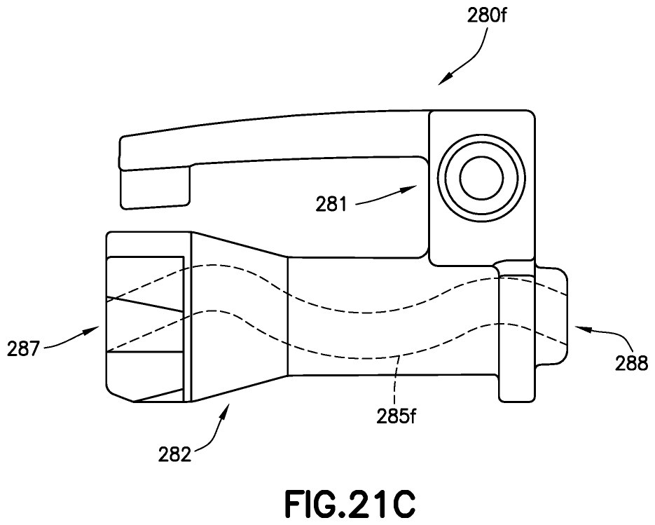

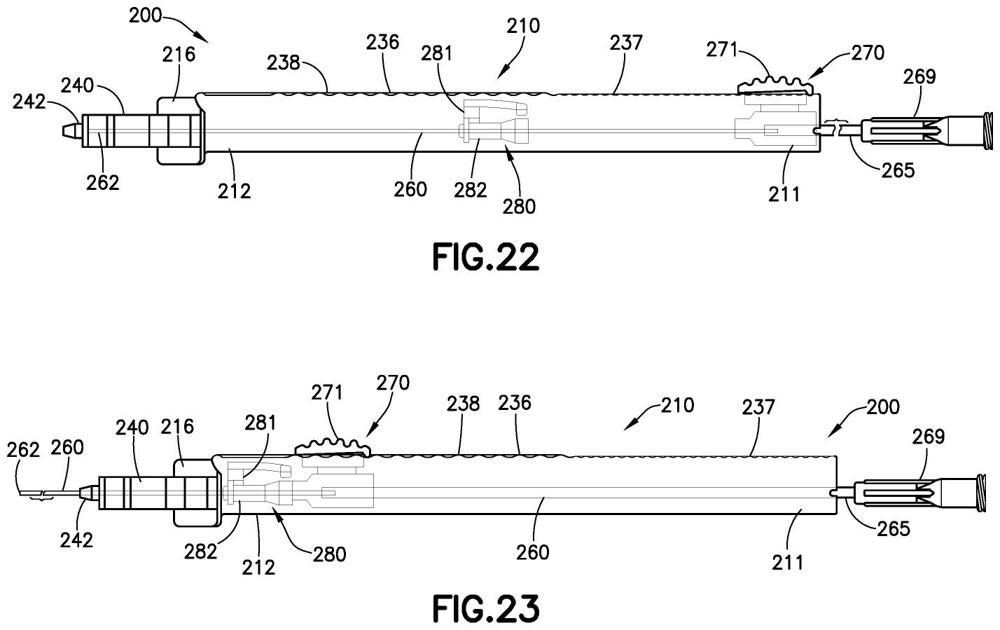

FIG. 16C is a rear view of the catheter support of FIG. 16A;

FIG. 16D is a front view of the catheter support of FIG. 16A;

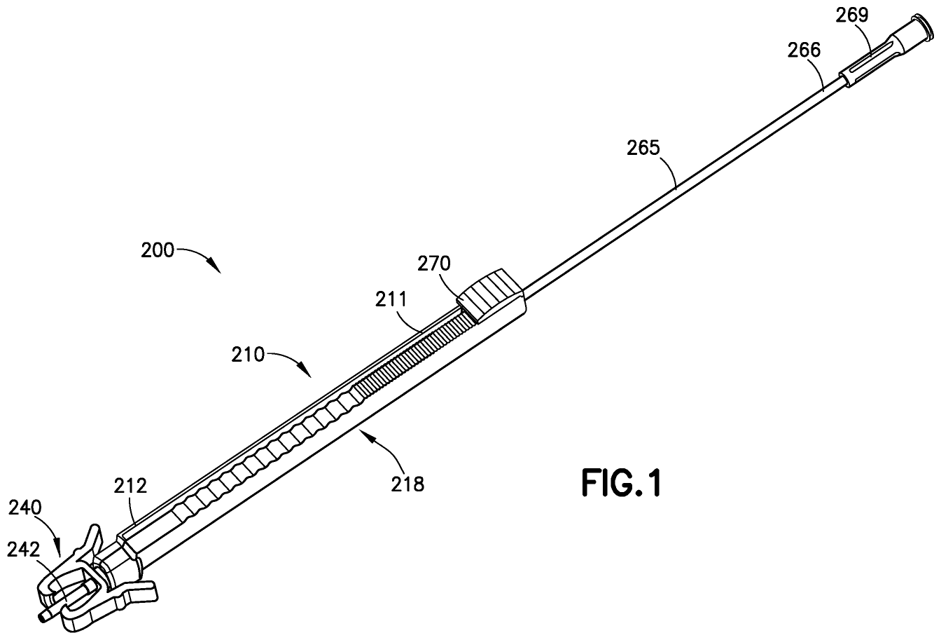

FIG. 17A is a rear perspective view of a catheter support that may be included in the fluid transfer device of FIG. 1, in accordance with another embodiment of the invention;

FIG. 1713 is a front perspective view of the catheter support of FIG. 17A;

FIG. 17C is a rear view of the catheter support of FIG. 17A;

FIG. 171) is a front view of the catheter support of FIG. 17A;

FIG. 18A is a front view of a catheter support that may be included in the fluid transfer device of FIG. 1, in accordance with another embodiment of the invention;

FIG. 18B illustrates the catheter support of FIG. 18A positioned in the introducer of FIG. 1 relative to the actuator and housing distal end;

FIG. 19A is a front view of a catheter support that may be included in the fluid transfer device of FIG. 1, in accordance with another embodiment of the invention;

FIG. 19B illustrates the catheter support of FIG. 19A positioned in the introducer of FIG. 1 relative to the actuator and housing distal end;

FIG. 20A is a rear perspective view of a catheter support that may be included in the fluid transfer device of FIG. 1, in accordance with another embodiment of the invention;

FIG. 20B is a front perspective view of the catheter support of FIG. 20A;

FIG. 20C is a side view of the catheter support of FIG. 20A, showing a passageway of the catheter support in phantom;

FIG. 21A is a rear perspective view of a catheter support that may be included in the fluid transfer device of FIG. 1, in accordance with another embodiment of the invention;

FIG. 21B is a front perspective view of the catheter support of FIG. 21A;

FIG. 21C is a side view of the catheter support of FIG. 21A, showing a passageway of the catheter support in phantom;

FIG. 22 is a side view of the blood draw device of FIG. 1 in a first configuration, showing positioning of the actuator and catheter support; and

FIG. 23 is a side view of the blood draw device of FIG. 1 in a second configuration, showing positioning of the actuator and catheter support.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

The present invention is directed to a fluid transfer device for phlebotomy through a peripheral intravenous line or catheter and a method of using the fluid transfer device to advance a flexible tube or catheter into the peripheral intravenous line and subsequently draw blood or administer a drug to a patient.

FIGS. 1-21 illustrate a fluid transfer device 200 and components thereof according to embodiments of the invention. The fluid transfer device 200 can be any suitable shape, size, or configuration and can be coupled to a PIVC (not shown in FIGS. 1-21), for example, via a lock and/or adapter. As described in further detail herein, a user can transition the fluid transfer device 200 from a first configuration to a second configuration to advance a catheter through an existing, placed, and/or indwelling PIVC (i.e., when the fluid transfer device 200 is coupled thereto) such that at least an end portion of the catheter is disposed in a distal position relative to the PIVC. Moreover, with peripheral intravenous lines each having a shape, size, and/or configuration that can vary based on, for example, a manufacturer of the PIVC and/or its intended usage, the fluid transfer device 200 can be arranged to allow the fluid transfer device 200 to be coupled to a PIVC having any suitable configuration and subsequently, to advance at least a portion of a catheter through the PIVC. In addition, the fluid transfer device 200 can be manipulated by a user to place a distal surface of the catheter a predetermined and/or desired distance beyond a distal surface of the PIVC to be disposed within a portion of a vein that receives a substantially unobstructed flow of blood.

As shown in FIGS. 1-3, the fluid transfer device 200 includes an introducer 210, a lock 240, a catheter 260, a secondary catheter 265, an actuator 270, and a catheter support 280. The introducer 210 can be any suitable shape, size, or configuration. For example, in some embodiments, the introducer 210 can be an elongate member having a substantially circular cross-sectional shape. In some embodiments, the shape of the introducer 210 and/or one or more features or surface finishes of at least an outer surface of the introducer 210 can be arranged to increase the ergonomics of the fluid transfer device 200, which in some instances, can allow a user to manipulate the fluid transfer device 200 with one hand (i.e., single-handed use).

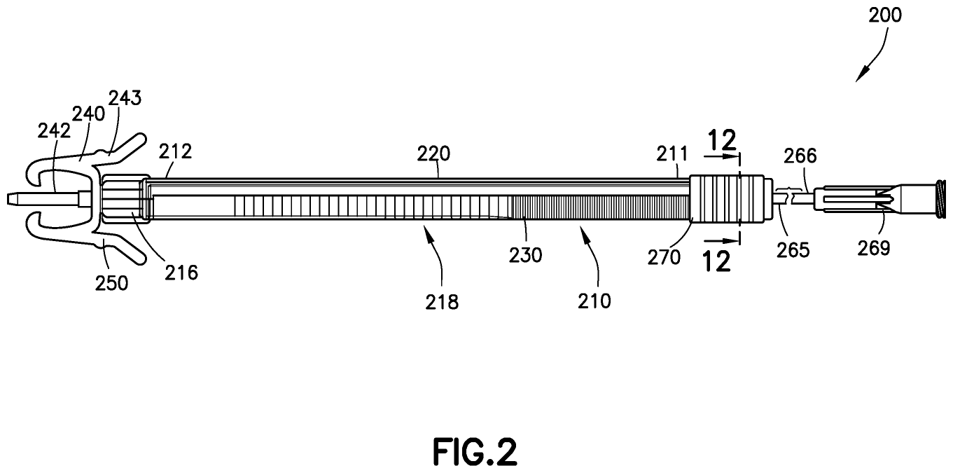

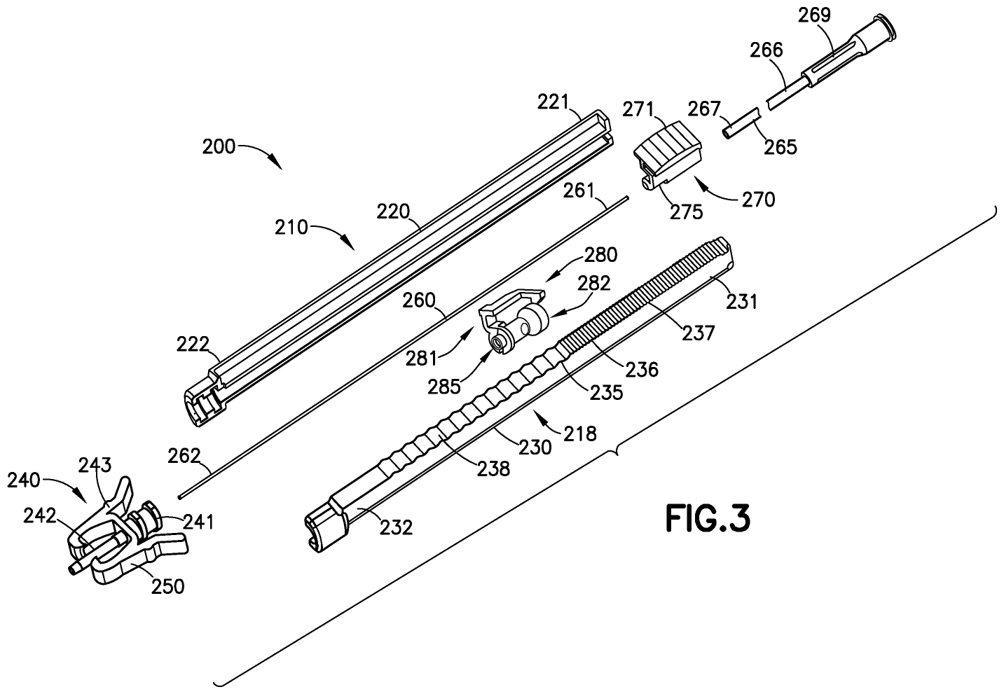

As shown in FIGS. 2-8, the introducer 210 of the fluid transfer device 200 includes an introducer housing 218 having a first housing member 220 and a second housing member 230 that are coupled to collectively form the housing 218. The first housing member 220 includes a proximal end portion 221, a distal end portion 222, and an inner surface 223. The inner surface 223 has a first portion 224 and a second portion 225, with second portion 225 being a flange in one embodiment. The proximal end portion 221 of the first housing member 220, and more specifically, a proximal wall of the first housing member 220 defines a notch 226 configured to selectively receive a portion of the secondary catheter 265, as described in further detail herein.

The second housing member 230 has a proximal end portion 231, a distal end portion 232, an inner surface 233, and a top surface 235. As described above with reference to the first housing member 220, the proximal end portion 231 of the second housing member 230, and more specifically, a proximal wall of the second housing member 230 defines a notch 234 configured to selectively receive a portion of the secondary catheter 265.

The top surface 235 of the second housing member 230 includes a set of ribs 236 distributed along a length of the second housing member 230, with each rib 236 extending along a width of the second housing member 230. The ribs 236 formed by the top surface 235 of the second housing member 230 can be any suitable shape, size, and/or configuration. For example, the set of ribs 236 includes a first portion 237 having a first size and shape, and a second portion 238 having a second size and shape, different from the first size and shape. Each rib in the second portion 238 has a size and shape that is greater than the size and shape of each rib of the first portion 237.

As described in further detail herein, a portion of the actuator 270 is configured to be advanced along the top surface 235 forming the set of ribs 236 as a user moves the actuator 270 relative to the introducer housing 218, which in turn, vibrates the actuator 270 (and the catheter 260 coupled thereto). In some instances, this vibration can, for example, facilitate the advancing of the catheter 260 through a portion or the fluid transfer device 200, a portion of the PIVC, and/or a portion of the vasculature. In some instances, the greater size of the ribs of the second portion 238 can result in a larger amount of vibration as the actuator 270 is moved along the top surface 235 (as described above). In some instances, the greater size of the ribs of the second portion 238 can result in an increase in a force otherwise sufficient to move the portion of the actuator 270 along the top surface 235.

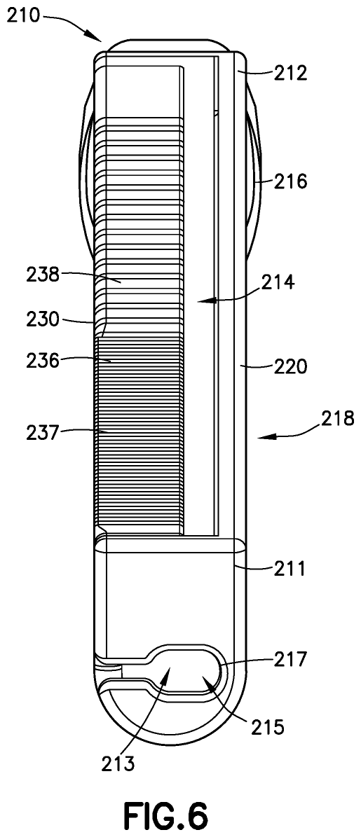

As shown in FIGS. 6-8, the first housing member 220 is configured to be coupled to the second housing member 230 to collectively form the introducer housing 218. In some embodiments, forming the introducer housing 218 from the first housing member 220 and the second housing member 230 can reduce undesirable variations in the shape and/or size of the inner surface 223 and 233 (e.g., due to draft angles and/or manufacturing tolerances) during manufacturing, which in some instances, can reduce a likelihood of kinks, bends, and/or deformations of the catheter 260 during use of the fluid transfer device 200. In some embodiments, forming the introducer housing 218 from the first housing member 220 and the second housing member 230 can allow at least the inner surface 223 of the first housing member 220 to form a tortuous shape that would otherwise present challenges when manufacturing the introducer housing 218 from a single work piece.

The first housing member 220 and the second housing member 230 collectively form a proximal end portion 211 and a distal end portion 212 of the introducer housing 218 and collectively define an inner volume 213 of the introducer housing 218. As shown in FIG. 6, the proximal end portion 211 of the introducer housing 218 defines an opening 217. Specifically, the opening 217 is collectively formed and/or defined by the notch 226 of the first housing member 220 and the notch 234 of the second housing member 230. The arrangement of the proximal end portion 211 is such that a portion of the opening 217 defined by the notch 226 of the first housing member 220 has a first size and/or shape and a portion of the opening 217 defined by the notch 234 of the second housing member 230 has a second size and/or shape that is less than the first size and/or shape. In other words, a portion of the opening 217 is constricted, pinched, obstructed, and/or otherwise reduced. As described in further detail herein, the opening 217 is configured to receive a portion of the secondary catheter 265, which can be moved within the opening 217 from the larger portion of the opening 217 to the reduced portion of the opening 217 (e.g., the portion formed by the notch 234 of the second housing member 230) to obstruct, pinch, and/or clamp the secondary catheter 265.

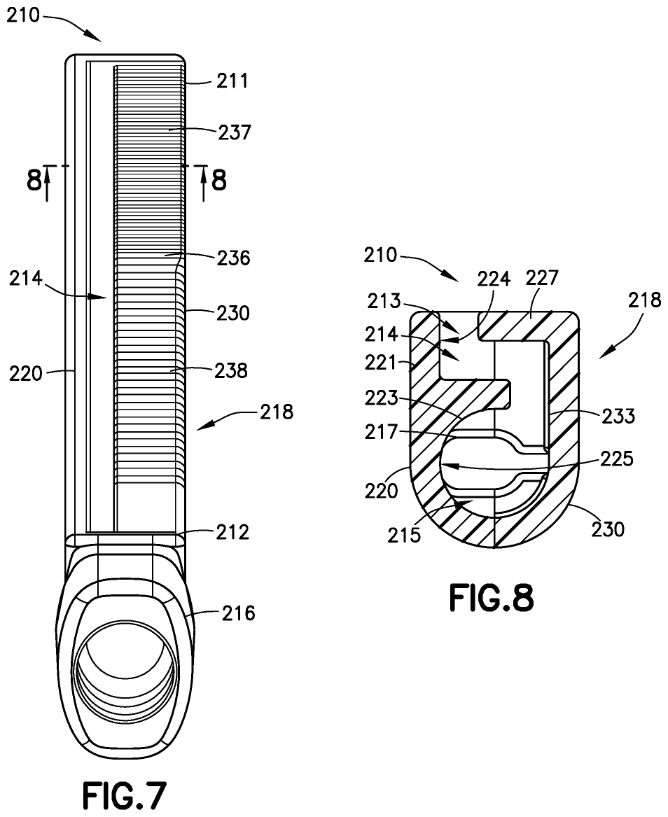

As shown in FIG. 7, the distal end portion 212 of the introducer housing 218 includes and/or otherwise forms a coupler 216. In other words, the distal end portion 222 of the first housing member 220 and the distal end portion 232 of the second housing member 230 collectively form the coupler 216 at the distal end portion 212 of the introducer housing 218. The coupler 216 can be any suitable shape, size, and/or configuration. For example, in this embodiments, the coupler 216 forms a set of threads, which can form a threaded coupling with an associated threaded portion of the lock 240, as described in further detail herein. Although not shown in FIG. 7, the distal end portion 211 of the introducer housing 218 can include and/or can be configured to receive a seal (not shown) that can selectively seal and/or fluidically isolate the inner volume 213 of the introducer housing 218 (at least from an open portion of the coupler 216). In use, the seal can be transitioned from a sealed or closed configuration to an open configuration to allow, for example, a portion of the catheter 260 to pass therethrough. In some embodiments, the seal can contact an outer surface of the catheter 260 to define a seal therebetween that is operable to limit and/or substantially prevent a back flow of fluid between the outer surface of the cannula and the seal.

The inner surface 223 of the first housing member 220 and the inner surface 233 of the second housing member 230 collectively define the inner volume 213 of the introducer housing 218. As shown in FIG. 8, the arrangement of the inner surfaces 223 and 233 is such that the inner volume 213 has and/or defines a tortuous cross-sectional shape. For example, the inner volume 213 can have a substantially S-shaped or an at least partially S-shaped cross-sectional shape. More specifically, the inner surface 223 of the first housing member 220 includes and/or forms a ridge, tab, flange, protrusion, and/or wall configured to separate the first portion 224 of the inner surface 223 from the second portion 225 of the inner surface 223. Thus, the tortuous cross-sectional shape of the inner volume 213 forms and/or defines a first portion 214 of the inner volume 213 and a second portion 215 of the inner volume 213. In this manner, the first portion 214 of the inner volume 213 is spaced apart from the second portion 215 of the inner volume 213 without being fluidically isolated therefrom. In other words, the first portion 214 of the inner volume 213 defines an axis that is parallel to and offset from an axis defined by the second portion 215 of the inner volume 213.

As shown in FIG. 8, the first portion 214 of the inner volume 213 extends through a wall of the introducer housing 218. Similarly stated, the introducer housing 218 defines (e.g., the first housing member 220 and the second housing member 230 collectively define) a slot, channel, track, opening, and/or the like that is in fluid communication with the first portion 214 of the inner volume 213. Conversely, the second portion 215 of the inner volume 213 is entirely defined and/or enclosed (at least in the circumferential direction) by the introducer housing 218. The tortuous cross-sectional shape of the inner volume 213 is such that the second portion 215 cannot be viewed (e.g., is out of the line of sight) via the slot (in fluid communication with the first portion 214 of the inner volume 213), which in turn, can limit and/or substantially prevent contamination of the catheter 260 disposed therein.

In this embodiment, the second portion 215 of the inner volume 213 is substantially aligned with, for example, a portion of the opening 217 and a portion of an opening defined by the coupler 216. Moreover, the second portion 215 of the inner volume 213 is configured to be substantially aligned with the lock 240 when the lock is coupled to the coupler 216 of the introducer housing 218. In other words, the axis defined by the second portion 215 of the inner volume 213 is substantially co-axial with an axis defined by a portion of the lock 240, as described in further detail herein. In this manner, the second portion 215 of the inner volume 213 can movably receive, for example, a portion of the actuator 270 and a portion of the catheter 260. Thus, the actuator 270 can be moved relative to the introducer housing 218 to move the catheter 260 between a first position, in which the catheter 260 is entirely disposed within the second portion 215 of the inner volume 213, and a second position, in which at least a portion of the catheter 260 extends outside of the second portion 215 of the inner volume 213 and distal to the introducer housing 218, as described in further detail herein.

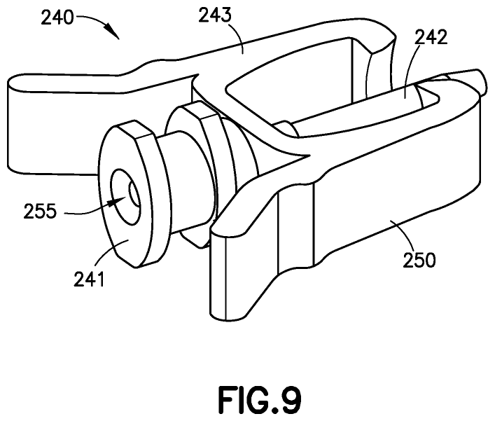

The lock 240 of the fluid transfer device 200 can be any suitable shape, size, and/or configuration. As described above, the lock 240 is configured to be physically and fluidically coupled to the introducer housing 218 and configured to couple the introducer 210 to the PIVC and/or any suitable intermediate device or adapter coupled to the PIVC. The lock 240 has a coupler 241, a blunt cannula 242, a first arm 243, and a second arm 250, as shown in FIG. 9. In addition, the lock 240 defines a lumen 255 extending through the coupler 241 and the blunt cannula 242. The coupler 241 is configured to couple the lock 240 to the coupler 216 of the introducer housing 218. Specifically, in this embodiment, the coupler 241 includes and/or forms one or more protrusions configured to selectively engage the threads defined and/or formed by the coupler 216 of the introducer housing 218, thereby forming a threaded coupling.

The blunt cannula 242 extends from the coupler 246 and is disposed between the first arm 243 and the second arm 250. The blunt cannula 242 can be any suitable shape, size, and/or configuration. In some embodiments, the blunt cannula 242 can have a length that is sufficient to extend through at least a portion of the PIVC or through an adapter and at least partially into or through the PIVC. Furthermore, the blunt cannula 242 can have an inner diameter (a diameter of a surface at least partially defining the lumen 255) that is similar to or slightly larger than an outer diameter of a portion of the catheter 260. Thus, the lumen 255 of the lock 240 can receive a portion of the catheter 260 when the fluid transfer device 200 is transitioned between the first configuration and the second configuration.

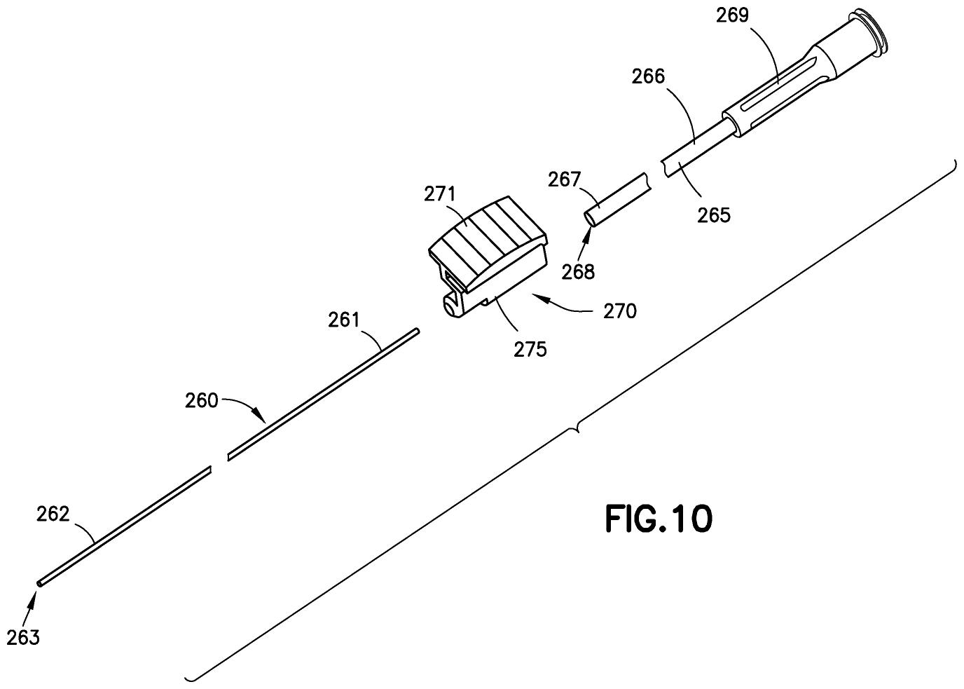

As described above, at least a portion of the catheter 260 and at least a portion of the secondary catheter 265 is movably disposed within the second portion 215 of the inner volume 213 defined by the introducer housing 218. As shown in FIG. 10, the catheter 260 has a proximal end portion 261 and a distal end portion 262 and defines a lumen 263. The proximal end portion 261 of the catheter 260 is coupled to a second portion 275 of the actuator 270. In this manner, the actuator 270 can be moved relative to the introducer housing 218 to move the catheter 260 between a first position, in which the catheter 260 is disposed within the introducer housing 218 (e.g., the entire catheter 260 is disposed within the introducer housing 218 or within the introducer housing 218 and the lock 240) and a second position, in which the distal end portion of the catheter 260 is at least partially disposed in a position distal to the lock 240 and/or the PIVC (not shown) when the lock 240 is coupled to the PIVC, as described in further detail herein. The distal end portion 262 can be any suitable shape, size, and/or configuration and can define at least one opening in fluid communication the lumen 263.

The secondary catheter 265 has a proximal end portion 266 and a distal end portion 267 and defines a lumen 268. A portion of the secondary catheter 265 is disposed within and extends through the opening 217 of the introducer housing 218 (e.g., collectively defined by the notches 223 and 233 of the first housing member 220 and second housing member 230, respectively). As such, the proximal end portion 266 is at least partially disposed outside of the introducer housing 218 and the distal end portion 267 is at least partially disposed within the second portion 215 of the inner volume 213 defined by the introducer housing 218. As described above, the secondary catheter 265 can be moved within the opening 217 between a first position and a second position to selectively clamp, pinch, kink, bend, and/or otherwise deform a portion of the secondary catheter 265, which in turn, obstructs, pinches, kinks, closes, seals, etc. the lumen 268 of the secondary catheter 265. For example, the first position can be associated and/or aligned with a first portion of the opening 217 having a larger perimeter and/or diameter than a perimeter and/or diameter of a second portion of the opening 217 associated and/or aligned with the second position. Thus, a user can manipulate the secondary catheter 265 to occlude the lumen 268 of the secondary catheter 265, thereby limiting, restricting, and/or substantially preventing a flow of a fluid therethrough.

As shown in FIG. 10, the proximal end portion 266 of the secondary catheter 265 is coupled to and/or otherwise includes a coupler 269. The coupler 269 is configured to physically and fluidically couple the secondary catheter 265 to any suitable device such as, for example, a fluid reservoir, fluid source, syringe, evacuated container holder (e.g., having a sheathed needle or configured to be coupled to a sheathed needle), pump, and/or the like. The distal end portion 267 of the secondary catheter 265 is at least partially disposed within the second portion 215 of the inner volume 213 defined by the introducer housing 218 and is coupled to a second portion 275 of the actuator 270. In some embodiments, the secondary catheter 265 can have a larger diameter than the catheter 260 such that the proximal end portion 261 of the catheter 260 is at least partially disposed within the lumen 268 defined by the secondary catheter 265 when the catheter 260 and the secondary catheter 265 are coupled to the second portion 275 of the actuator 270. In some embodiments, such an arrangement can, for example, reduce and/or substantially prevent leaks associated with fluid flowing between the catheter 260 and the secondary catheter 265. In some embodiments, such an arrangement can also limit, reduce, and/or substantially prevent hemolysis of a volume of blood as the volume of blood flows through the catheter 260 and the secondary catheter 265. In this manner, when the coupler 269 is coupled to a fluid reservoir, fluid source, syringe, evacuated container, pump, etc., the secondary catheter 265 establishes fluid communication between the reservoir, source, pump, etc. and the catheter 260.

The actuator 270 of the fluid transfer device 200 is coupled to the catheter 260 and can be moved along a length of the introducer housing 218 to transition the fluid transfer device 200 between its first configuration, in which the catheter 260 is in the first position, and its second configuration, in which the catheter 260 is in the second position. The actuator 270 can be any suitable shape, size, and/or configuration. For example, in some embodiments, the actuator 270 can have a size and shape that is associated with and/or based at least in part on a size and/or shape of the introducer housing 218.

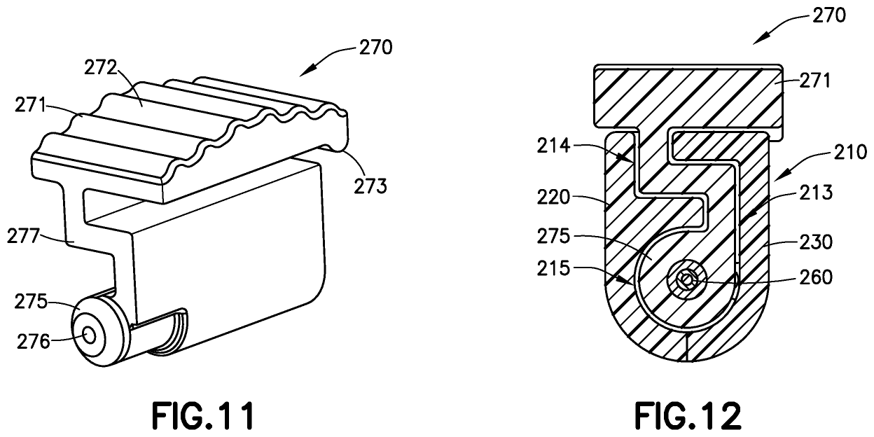

As shown in FIGS. 11 and 12, the actuator 270 includes a first portion 271, the second portion 275, and a wall 277 extending therebetween. The first portion 271 of the actuator 270 is at least partially disposed within the first portion 214 of the inner volume 213 defined by the introducer housing 218 and the second portion 275 of the actuator 270 is disposed within the second portion 215 of the inner volume 213, as described above. The first portion 271 of the actuator 270 includes an engagement member 272. The arrangement of the actuator 270 is such that the engagement member 272 is disposed outside of the introducer housing 218 while the rest of the first portion 271 is within the first portion 214 of the inner volume 213 defined by the introducer housing 218. As such, the engagement member 272 can be engaged and/or manipulated by a user (e.g., by a finger or thumb of the user) to move the actuator 270 relative to the introducer housing 218. In some embodiments, the engagement member 272 can include a set of ridges and/or any suitable surface finish that can, for example, increase the ergonomics of the actuator 270 and/or fluid transfer device 200.

The engagement member 272 includes a tab 273 disposed at or near a proximal end portion of the engagement member 272. The tab 273 can be any suitable tab, rail, ridge, bump, protrusion, knob, roller, slider, etc. that extends from a surface of the engagement member 272. The tab 273 is configured to selectively engage the top surface 235 of the second housing member 230 of the introducer housing 218. More specifically, the tab 273 is in contact with the ribs 236 formed by the second housing member 230 and moves along each successive rib as the actuator 270 is moved along a length of the introducer housing 218, with interaction of the tab 273 with the ribs 236 causing vibration of the actuator 270.

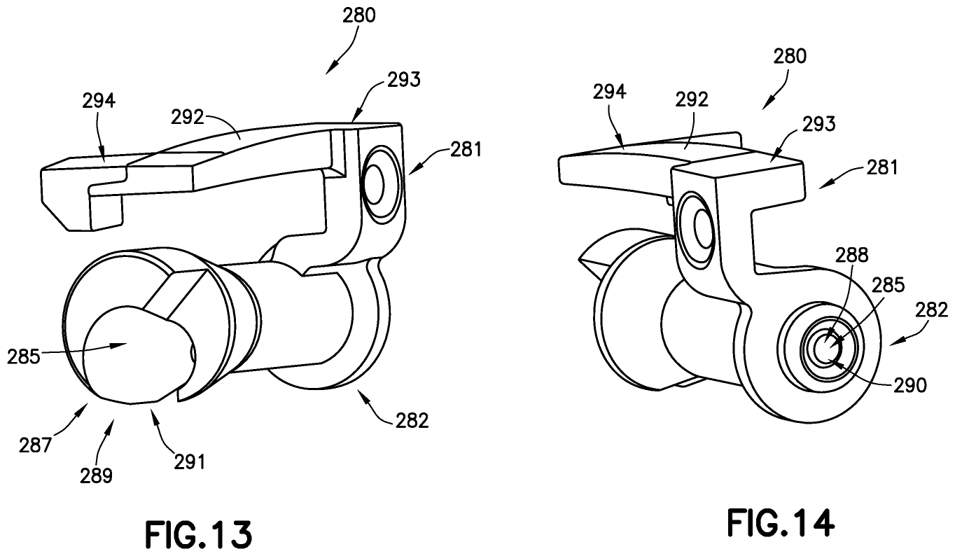

As shown in FIG. 3 and now also in FIGS. 13-15, the catheter support 280 (or alternately “support member”) of introducer 210 generally includes a bracket portion 281 and a hub portion 282. The bracket portion 281 of the catheter support 280 is at least partially disposed within the first portion 214 of the inner volume 213 defined by the introducer 210 and the hub portion 282 of the catheter support 280 is disposed within the second portion 215 of the inner volume 213.

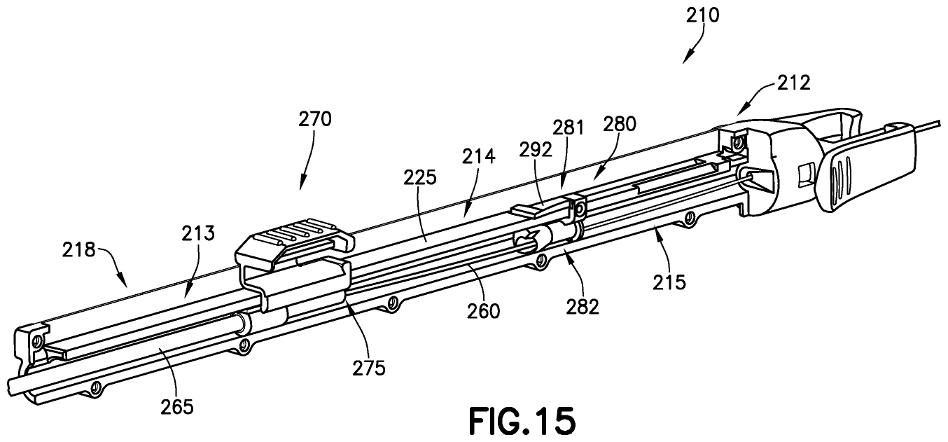

In some embodiments, and as illustrated in FIGS. 3 and 15, the bracket portion 281 of the catheter support 280 is entirely disposed within the first portion 214 of the inner volume 213 defined by the introducer 210. In other non-illustrated embodiments, the bracket portion 281 of the catheter support 280 may optionally include an engagement member that is disposed outside of the introducer 210 so as to be engaged and/or manipulated by a user, for example, by a finger or thumb of the user, to move the catheter support 280 relative to the introducer 210.

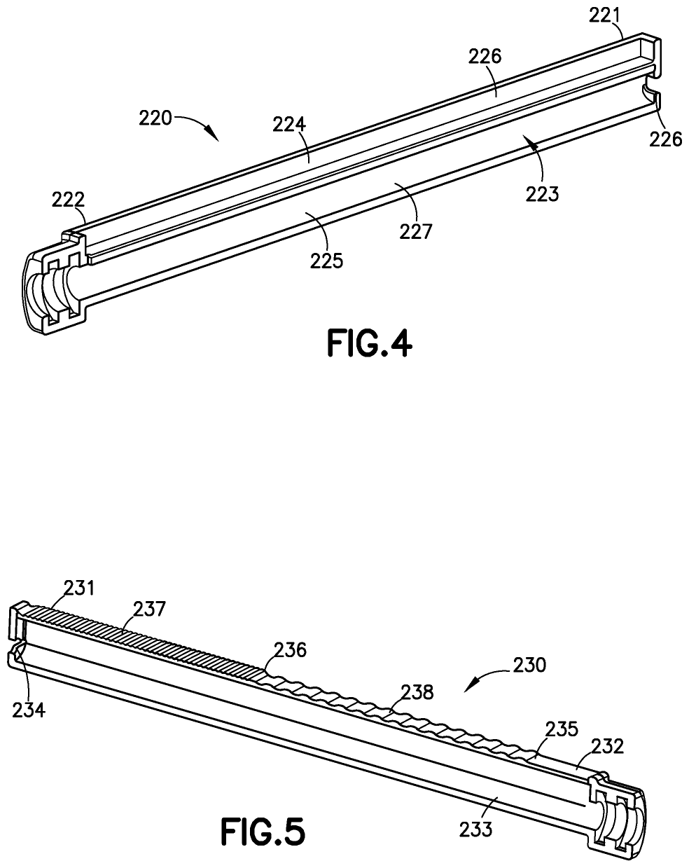

The hub portion 282 has a cross-sectional shape that is based at least in part on a cross-sectional shape of the second portion 215 of the inner volume 213 defined by the introducer 210, for example, at least a partially circular cross-sectional shape. In this manner, the inner surface 223 of the first member 220 (FIG. 4) and the inner surface 233 of the second member 230 (FIG. 5) can support and/or guide the hub portion 282 of the catheter support 280 as the catheter support 280 is moved relative to the introducer 210.

The hub portion 282 defines a passageway 285 having a proximal opening 287 on the proximal end of the hub portion 282 and a distal opening 288 on the distal end of the hub portion 282. The passageway 285 is sized and shaped to allow the catheter 260 to freely pass through the passageway 285 while being at least partially contained to prevent excessive movement of the catheter 260 within the second portion 215 of the inner volume 213 of the introducer 210. Additionally, the passageway 285 is configured and shaped to bias or allow a downward bowing of the catheter 260, so as to minimize the chance of the catheter 260 buckling as the actuator 270 is advanced toward the distal end portion 212 of the introducer 210, as will be explained in more detail below.

The passageway 285 may comprise two portions—with a proximal portion 289 adjacent to the proximal opening 287 and a distal portion 290 adjacent to the distal opening 288. The proximal portion 289 of the passageway 285 may be sized and shaped to receive at least a portion of the second portion 275 of the actuator 270, such as the proximal portion 289 being substantially cone-shaped or funnel-shaped. As shown in FIG. 21, a notch 291 may be provided in the cone-shaped or funnel-shaped proximal portion 289 of the passageway 285.

The bracket portion 281 may have a tortuous cross-sectional shape that is based at least in part on the tortuous cross-sectional shape of the inner volume 213 of the introducer 210. In this manner, the bracket portion 281 of the catheter support 280 can define an axis that is parallel to but offset from an axis defined by the hub portion 282 of the catheter support 280. For example, the bracket portion 281 can have a substantially S-shaped or an at least partially S-shaped cross-sectional shape, or the bracket portion 281 can form dogleg shape or the like. The tortuous cross-sectional shape of the bracket portion 281 and thus, the catheter support 280, may be such that the hub portion 282 of the catheter support 280 cannot be viewed via the first portion 214 of the inner volume 213 and is out of the line of sight of the first portion 214 of the inner volume 213 defined by the introducer 210.

At least a portion of the bracket portion 281 of the catheter support 280 has a profile corresponding to an outer surface of the flange 225 provided on the inner surface of the introducer 210, such that the bracket portion 281 fits over and at least partially covers the flange 225 and is movable with respect to the introducer 210 in a direction from the proximal end portion 211 of the introducer 210 to the distal end portion 212 of the introducer 210 and vice versa along the flange 225. When the catheter support 280 is moved with respect to the introducer 210, the bracket portion 281 moves within the first portion 214 of the inner volume 213, and the hub portion 282 moves within the second portion 215 of the inner volume 213.

In the non-limiting illustrated embodiments, the bracket portion 281 of the catheter support 280 includes a cantilever arm 292 that is biased against the flange 225 of the introducer 210. The cantilever arm 292 includes a first end 293 extending from the hub portion 282 of the catheter support 280 and a second end 294 opposite the first end 293. The cantilever arm 292 may be molded in a curved down shape or angled down, such that the second end 294 is lower than the first end 293. The gap between the hub portion 282 and the bracket portion 281 is small enough that the cantilever arm 292 pinches the flange 225 of the introducer 210 that it wraps around. This provides friction between the introducer 210 and the catheter support 280 such that the catheter support 280 will not move under gravity but only when pushed on by assembly tooling or the actuator 270.

The catheter support 280 of introducer 210 is positioned within the introducer housing 218 at a location between the actuator 270 and the distal end 212 of the introducer 210. The catheter 260 is routed through the passageway 285 of the hub portion 282 to reduce an unsupported length of the catheter 260, effectively increasing the resistance the catheter 260 can withstand before buckling. That is, the force at which a catheter 260 buckles is inversely proportional to the effective length squared. Thus, as the effective length of the catheter 260 increases, the buckling force required to buckle the catheter 260 decreases, making the catheter more prone to buckling. The catheter support 280 supports the catheter 260, thereby shortening the effective length of the catheter 280, increasing the force required to buckle the catheter 260, and decreasing the tendency of the catheter 260 to buckle—i.e., the catheter support 280 divides the unsupported length of the catheter 260 into two smaller portions that are more resistant to bending, kinking, flexing, and/or deformation.

While the catheter support 280 is positioned to reduce the unsupported length of the catheter 260 and increase the resistance thereof to buckling, it is recognized that bowing of the catheter 260 can still occur when a load is applied to the catheter 260 responsive to movement of the actuator 270 toward the distal end 212 of introducer 210. As the catheter 260 is offset vertically downward within the introducer housing 218 (in order to accommodate the second portion 275 of the actuator 270 within the second portion 215 of the inner volume 213), the upper clearance between the housing 218 and the catheter 260 is much larger than the lower clearance, and the catheter 260 tends to bow upward when a high enough load is applied thereto. When the catheter 260 bows upwards, any additional force applied to the actuator 270 by a user will serve more to deform and buckle the catheter 260 than push it forwards, as compared to if the catheter 260 were to bow downwards, then the force applied to the actuator 270 will serve more to advance the catheter 260 than deform the catheter.

In view of the above, the catheter support 280 may be configured in a manner that encourages a downward bowing of the catheter 260 in the catheter support during operation of the introducer 210 and/or pre-biases a downward bowing of the catheter 260. FIGS. 16A-16D, 17A-17D, 18A and 18B, 19A and 19B, 20A-20C and 21A-21C illustrate embodiments of a catheter support 280 that provide for such downward deflection biasing of the catheter 260, so as to provide for advancement of the catheter 260 into the PIVC while minimizing the potential for buckling thereof.

Referring to FIGS. 16A-16D, a catheter support 280a is shown that encourages a downward bowing of the catheter 260, according to an embodiment. As previously described, the catheter support 280a generally includes a bracket portion 281 and a hub portion 282. The hub portion 282 includes a top surface 300 (from which bracket portion 281 extends upwardly) and a bottom surface 301 and defines a passageway 285 having a proximal opening 287 on the proximal end of the hub portion 282 and a distal opening 288 on the distal end of the hub portion 282.

As shown in FIGS. 16A-16D, the passageway 285 is formed as an open slotted passageway 285a. The open slotted passageway 285a is formed between the proximal opening 287 and the distal opening 288 and may generally be described as including a top portion 302 and a bottom portion 303. The top portion 302 of passageway is generally aligned with distal opening 288 and with a center axis 304 of the hub portion 282 and a path along which the length of the catheter 260 extends between the actuator 270 and the distal end portion 212 of the introducer 210. The bottom portion 303 of passageway is vertically below the top portion 302 and extends down to the bottom surface 301 of the hub portion 282, such that the slotted passageway 285a is open along the bottom surface 301 of the hub portion 282.

As previously indicated, the catheter 260 is routed through the passageway 285 of the hub portion 282 to reduce an unsupported length of the catheter 260. In the present embodiment, the catheter 260 is received in the top portion 302 of the open slotted passageway 285a and is retained therein during normal operation of the introducer 210. However, during periods of operation of the introducer 210 where a large load is applied to the catheter 260—such as when the distal end portion 262 of the catheter 260 reaches a curved region of the PIVC and the actuator 270 is urged further forward toward the distal end 212 of introducer 210—the catheter 260 may begin to deflect or bow. During such a loading event, the construction of open slotted passageway 285a allows for the catheter 260 to bow downward within the open slotted passageway 285a, from the top portion 302 down toward the bottom portion 303. That is, as the top and side surfaces surrounding the top portion 302 of passageway 285a are closed, the catheter 260 is only allowed to bow downward at the location of the catheter support 280 when a load is applied thereto. As previously described, a downward bowing of the catheter 260 minimizes the potential for buckling thereof to occur as compared to if the catheter 260 were to bow upward. Accordingly, the catheter support 280a minimizes the potential for buckling of the catheter 260 during use of the fluid transfer device 200.

Referring to FIGS. 17A-17D, another embodiment of a catheter support 280b is shown that encourages a downward bowing of the catheter 260. Similar to the catheter support of FIGS. 16A-16D, the catheter support 280b includes a slotted passageway formed in the hub portion 282 that extends between the proximal opening 287 and the distal opening 288. However, the slotted passageway in the catheter support 280b is a closed slotted passageway 285b. The closed slotted passageway 285b includes a top portion 302 and a bottom portion 303. The top portion 302 of passageway is generally aligned with distal opening 288 and with the center axis 304 of the hub portion 282 and the path along which the length of the catheter 260 extends between the actuator 270 and the distal end portion 212 of the introducer 210. The bottom portion 303 of passageway is vertically below the top portion 302 and extends downwardly from the top portion 302—ending at a location that is vertically between the center axis 304 and the bottom surface 301 of the hub portion 282 (i.e., the bottom portion 303 of slotted passageway is “closed”).

With catheter support 280b, the catheter 260 is received in the top portion 302 of the closed slotted passageway 285b and is retained therein during normal operation of the introducer 210. However, during periods of operation of the introducer 210 where a large load is applied to the catheter 260—such as when the distal end portion 262 of the catheter 260 reaches a curved region of the PIVC and the actuator 270 is urged further forward toward the distal end 212 of introducer 210—the catheter 260 may begin to deflect or bow. During such a loading event, the construction of closed slotted passageway 285b allows for the catheter 260 to bow downward within the closed slotted passageway 285b, from the top portion 302 down toward the bottom portion 303. That is, as the top and side surfaces surrounding the top portion 302 of passageway are closed, the catheter 260 is only allowed to bow downward at the location of the catheter support 280 when a load is applied thereto. While the catheter 260 is urged to bow downward at the location of the catheter support 280, the structure of the closed slotted passageway 285b ensures that the catheter 260 is retained within the passageway. Accordingly, the catheter support 280b minimizes the potential for buckling of the catheter 260 during use of the fluid transfer device 200.

While each of the catheter supports 280a, 280b has a slotted passageway formed therein where the top portion 302 is generally aligned with the center axis 304 of the hub portion 282 and with the path along which the length of the catheter 260 extends between the actuator 270 and the distal end portion 212 of the introducer 210, additional embodiments of a catheter support may be structured to include a slotted passageway where openings 287, 288 and the top portion 302 of the passageway are vertically offset from the center axis 304 and catheter path. That is, catheter supports 280c, 280d are configured where the slotted passageway (whether an open slotted passageway or closed slotted passageway) is shifted downward in the hub portion 282.

Referring to FIGS. 18A and 18B, a catheter support 280c is shown as including an open slotted passageway 285c formed within hub portion 282, where the openings 287, 288 and top portion 302 of the passageway are offset vertically downward from the center axis 304 and from a catheter path along which the length of the catheter 260 extends between the actuator 270 and the distal end portion 212 of the introducer 210. With the distal opening 288 and the open slotted passageway 285c being offset vertically downward from the normal catheter path of catheter 260, the catheter 260 is biased in the downward direction as it is retained in top portion 302—and this being prior to any load being applied to the catheter 260. This pre-biasing of the catheter 260 to have a downward bowing minimizes the potential for buckling of the catheter 260. Additionally, the structure of the open slotted passageway 285c allows for still additional downward bowing of the catheter 260 at the location of the catheter support 280c as a load is applied thereto, to provide another means of minimizing the potential for buckling of the catheter 260.

Referring to FIGS. 19A and 19B, a catheter support 280d is shown as including a closed slotted passageway 285d formed within hub portion 282, where the openings 287, 288 and the top portion 302 of the passageway are offset vertically downward from the center axis 304 and from a catheter path along which the length of the catheter 260 extends between the actuator 270 and the distal end portion 212 of the introducer 210. As described above, with the distal opening 288 and the closed slotted passageway 285d being offset vertically downward from the normal catheter path of catheter 260, the catheter 260 is biased in the downward direction as it is retained in top portion 302—and this being prior to any load being applied to the catheter 260. This pre-biasing of the catheter 260 to have a downward bowing minimizes the potential for buckling of the catheter 260, and the structure of the closed slotted passageway 285d allows for still additional downward bowing of the catheter 260 at the location of the catheter support 280 as a load is applied thereto, to provide another means of minimizing the potential for buckling of the catheter 260.

Referring now to FIGS. 20A-20C, another embodiment of a catheter support 280e is shown that encourages a downward bowing of the catheter 260. As previously described, the catheter support 280e generally includes a bracket portion 281 and a hub portion 282, with the hub portion 282 having a passageway 285 with a proximal opening 287 and a distal opening 288. In the

As shown in FIGS. 20A-20C, the passageway 285 is formed as a non-linear, curved or arcuate passageway 285e. The arcuate passageway 285e is formed to curve upwardly within hub portion 282, with a central region of the passageway (between the proximal and distal openings 287, 288 of the hub portions 282) being offset vertically upward from the proximal opening 287 and distal opening 288 of the passageway. The arcuate passageway 285e may have a generally cylindrical profile to accommodate routing of the catheter 260 therethrough and provide for movement of the catheter 260. The proximal opening 287 and distal opening 288 of the passageway may be generally aligned with the center axis 304 of the hub portion 282 and with the path along which the length of the catheter extends between the actuator 270 and the distal end portion 212 of the introducer 210.

As previously indicated, the catheter 260 is routed through the passageway 285 of the hub portion 282 to reduce an unsupported length of the catheter 260. With the curved passageway 285e having a central region that is offset vertically upward from the normal catheter path of catheter 260, the catheter 260 is caused to follow the passageway 285e in the upward direction, and is biased in the downward direction as it enters into the proximal opening 287 of the hub portion 282 and exits the distal opening 288 of the hub portion 282. This pre-biasing of the catheter 260 to have a downward bowing minimizes the potential for buckling of the catheter 260 as a load is applied thereto. Accordingly, the catheter support 280e minimizes the potential for buckling of the catheter 260 during use of the fluid transfer device 200.

Referring now to FIGS. 21A-21C, another embodiment of a catheter support 280f is shown that encourages a downward bowing of the catheter 260. As previously described, the catheter support 280f generally includes a bracket portion 281 and a hub portion 282, with the hub portion 282 having a passageway 285 with a proximal opening 287 and a distal opening 288.

As shown in FIGS. 21A-21C, the passageway 285 is formed as a non-linear, waved or sinusoidal shaped passageway 285f. The sinusoidal passageway 285f is formed to curve vertically upward and downward within hub portion 282 as it runs between the proximal opening 287 and the distal opening 288 of the hub portion 282. The sinusoidal passageway 285f may have a generally cylindrical profile to accommodate routing of the catheter 260 therethrough and provide for movement of the catheter 260. The proximal opening 287 and distal opening 288 of the sinusoidal passageway 285f may be generally aligned with the center axis 304 of the hub portion 282 and with the path along which the length of the catheter extends between the actuator 270 and the distal end portion 212 of the introducer 210.

As previously indicated, the catheter 260 is routed through the passageway 285 of the hub portion 282 to reduce an unsupported length of the catheter 260. The sinusoidal passageway 285f causes the catheter 260 to curve vertically upward and downward as it passes through hub portion 282, and the catheter 260 is biased in the downward direction as it enters into the proximal opening 287 of the hub portion 282 and exits the distal opening 288 of the hub portion 282. This pre-biasing of the catheter 260 to have a downward bowing minimizes the potential for buckling of the catheter 260 as a load is applied thereto. Accordingly, the catheter support 280f minimizes the potential for buckling of the catheter 260 during use of the fluid transfer device 200.

Referring now to FIGS. 22 and 23, changing of the fluid transfer device 200 between the first configuration and second configuration is shown. The fluid transfer device 200 can be in the first configuration prior to use and can be transitioned by a user (e.g., a doctor, physician, nurse, technician, phlebotomist, and/or the like) from the first configuration (FIG. 22) to the second configuration (FIG. 23) to dispose at least the distal end portion 262 of the catheter 260 in a distal position relative to the introducer housing 28 (e.g. within an indwelling PIVC (not shown) or distal to the indwelling PIV).

The fluid transfer device 200 is in the first configuration of FIG. 22 when the catheter 260 is disposed in the first position within the introducer housing 218. In some embodiments, substantially the entire catheter 260 is disposed within the housing 218 when the catheter 260 is in the first position. In such embodiments, the introducer housing 218 can include a seal or the like (as described above) that can substantially seal the distal end portion 212 of the housing 210 to isolate the catheter 260 within the second portion 215 of the inner volume 213. In other embodiments, the catheter 260 is disposed within the housing 210 and the lock 240 when catheter 260 is in the first position.

With the fluid transfer device 200 in the first configuration, the actuator 270 is disposed toward the proximal end portion 211 of the introducer 210. The proximal end portion 261 of the catheter 260 is coupled to the second portion 275 of the actuator 270, and the catheter 260 extends through the second portion 215 of the inner volume 213 of the introducer 210 and through the passageway 285 of the catheter support 280, with the distal end portion 262 of the catheter 260 being received in the lumen of the coupler 216 and/or the lumen 255 of the lock 240.

The initial, pre-use position of the catheter support 280 may be set such that, in the first configuration, the position of the hub portion 282 along the unsupported portion of the catheter 260 (extending from the second portion 275 of the actuator 270 to the distal end portion 212 of the introducer 210) is at the center point of the unsupported portion of the catheter 260 or may be offset from the center point of the unsupported length of the catheter 260. For example, in the first configuration, the hub portion 282 of the catheter support 280 may be located in a position corresponding to a position of the actuator 270 when the advancement of the distal end portion 262 of the catheter 260 into the PIVC meets resistance requiring additional force to be provided to the actuator 270 to further advance the catheter 260—such as at a region where the catheter 260 enters the skin, curves in an upward direction, and curves again to pass along the vein.

To transition from the first configuration to the second configuration, the user engages the engagement member 272 of the first portion 271 of the actuator 270 to move the actuator 270 relative to the introducer housing 218, which in turn, moves the catheter 260 from the first position (e.g., disposed within the introducer housing 218) toward the second position. In this manner, the catheter 260 is moved through the second portion 215 of the inner volume 213, the passageway 285 of the hub portion 282 of the catheter support 280, and the lumen 255 of the lock 240 and, as such, at least the distal end portion 262 of the catheter 260 is disposed outside of and distal to the lock 240.

After partial movement of the actuator 270 in the distal direction and partial advancement of the catheter 260 towards the second position, the actuator 270 contacts the catheter support 280. In some embodiments, the distal end of the second portion 275 of the actuator 270 may abut the proximal end of the hub portion 282 or may be sized and shaped to be at least partially received within the proximal portion 289 of the passageway 285 of the hub portion 282. In other embodiments, a cam member (not shown) may be included on the distal end of the second portion 275 of the actuator 270 that contacts the cantilever arm 292 of the bracket portion 281 of the catheter support 280.

As the actuator 270 is advanced further in the distal direction, the actuator 270 pushes the hub portion 282 of the catheter support 280 in the distal direction and the catheter support 280 is advanced in the second portion 215 of the inner volume 213 of the introducer 210 until the catheter support 280 abuts the distal wall of the introducer 210, as shown in FIG. 23.

In some instances, the catheter 260 is considered to be in the second position when the actuator 270 and catheter support 280 are in a distal most position, with the distal end portion 262 of the catheter 260 thus being placed in a desired position relative to a distal end portion of the PIVC. In some instances, for example, a distal end 262 of the catheter 260 can be substantially flush with a distal end of the PIVC when the catheter 260 is in the second position. In other instances, the distal end 262 of the catheter 260 can extend a predetermined distance beyond the distal end of the PIVC (e.g., distal to the distal end of the PIVC), such that the distal end 262 of the catheter 260 is positioned within the vein at a predetermined distance beyond the distal surface of the catheter 260 (e.g., a position within a vein that is substantially free from debris (e.g., fibrin/blood clots) otherwise surrounding the distal end portion of the PIVC).

With the catheter 260 in the second position (e.g., with the fluid transfer device 200 in the second configuration as shown in FIG. 23), the user can establish fluid communication between a fluid reservoir, fluid source, syringe, and/or the like and the catheter 260. With the catheter 260 in fluid communication with the fluid reservoir and/or fluid source, the fluid transfer device 200 can then aspirate a volume of blood from the vein based at least in part on disposing the distal surface of the catheter 260 at the predetermined and/or desired distance beyond the distal surface of the PIVC. In some instances, once a desired amount of blood has been collected and/or once a desired volume of a drug has been delivered to the patient, the user can move the actuator 270 in the proximal direction, such as moving the actuator 270 back to its proximal most position, so as to move the catheter 260 back to the first position.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A fluid transfer device for advancing a catheter into an indwelling catheter of a vascular access device, the fluid transfer device comprising:
an introducer having a proximal end portion and a distal end portion, the distal end portion coupleable to the vascular access device, the introducer defining an inner volume configured to movably receive the catheter;
an actuator movably coupled to the introducer and configured to move relative to the introducer to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter; and
a catheter support movably coupled to the introducer, and positioned between the actuator and the distal end portion of the introducer, the catheter support defining a passageway therein that receives the catheter to provide support to a length of the catheter that extends between the actuator and the distal end portion of the introducer;
wherein the passageway in the catheter support is configured to encourage a downward bowing of the catheter in the catheter support and/or pre-bias a downward bowing of the catheter.

2. The fluid transfer device of claim 1, wherein the catheter support comprises a bracket portion and a hub portion, the hub portion including a top surface and a bottom surface, with a center axis of the hub portion extending lengthwise between a proximal end and a distal end of the hub portion.

3. The fluid transfer device of claim 2, wherein the passageway comprises:
a proximal opening and a distal opening;
an open slotted passageway extending between the proximal and distal openings, the open slotted passageway including:
a top portion generally aligned with the distal opening; and
a bottom portion vertically below the top portion and that is open along the bottom surface of the hub portion;
wherein the catheter is encouraged to bow downward within the open slotted passageway, from the top portion down toward the bottom portion.

4. The fluid transfer device of claim 2, wherein the passageway comprises:
a proximal opening and a distal opening;
a closed slotted passageway extending between the proximal and distal openings, the closed slotted passageway including:
a top portion generally aligned with the distal opening; and
a bottom portion vertically below the top portion, with the bottom portion ending at a location vertically between the center axis and the bottom surface of the hub portion;
wherein the catheter is encouraged to bow downward within the closed slotted passageway, from the top portion down toward the bottom portion.

5. The fluid transfer device of claim 3, wherein the proximal opening, the distal opening, and the top portion are aligned with a tubing path along which the length of the catheter extends between the actuator and the distal end portion of the introducer.

6. The fluid transfer device of claim 3, wherein the proximal opening, the distal opening, and the top portion are offset vertically downward from a tubing path along which the length of the catheter extends between the actuator and the distal end portion of the introducer, thereby preloading the catheter to force a downward bowing thereof.

7. The fluid transfer device of claim 3, wherein the catheter is allowed to only bow downward at a location of the catheter support, responsive to the actuator moving along the housing to advance the catheter from the first position toward the second position.

8. The fluid transfer device of claim 2, wherein the passageway comprises a curved passageway, with a center region of the passageway between the proximal end and the distal end of the hub portion being offset vertically upward from a proximal opening and a distal opening of the passageway, the curved passageway pre-biasing a downward bowing of the catheter.

9. The fluid transfer device of claim 2, wherein the passageway comprises a sinusoidal passageway that shifts vertically as it runs between the proximal end and the distal end of the hub portion, the sinusoidal passageway pre-biasing a downward bowing of the catheter.

10. The fluid transfer device of claim 2, wherein the proximal end of the hub portion is configured to engage the actuator, such that during movement of the actuator to move the catheter from the first position to the second position, the actuator contacts the proximal end to move the catheter support with respect to the introducer.

11. The fluid transfer device of claim 1, wherein the distal end portion of the introducer comprises a lock configured to couple the introducer to the vascular access device.

12. A fluid transfer device for advancing a catheter into an indwelling catheter of a vascular access device, the fluid transfer device comprising:
an introducer having a proximal end portion and a distal end portion, the distal end portion coupleable to the vascular access device, the introducer defining an inner volume configured to movably receive the catheter;
an actuator movably coupled to the introducer and configured to move relative to the introducer to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter; and
a catheter support movably coupled to the introducer, and positioned between the actuator and the distal end portion of the introducer, the catheter support comprising a bracket portion and a hub portion, the hub portion defining a passageway therein that receives the catheter to provide support to a length of the catheter that extends between the actuator and the distal end portion of the introducer;
wherein the passageway comprises:
a proximal opening and a distal opening;
a slotted passageway extending between the proximal and distal openings, the slotted passageway configured to encourage a downward bowing of the catheter within the slotted passageway.

13. The fluid transfer device of claim 12, wherein the slotted passageway comprises an open slotted passageway including:
a top portion generally aligned with the distal opening; and
a bottom portion vertically below the top portion, with the bottom portion ending at a location vertically between the center axis and the bottom surface of the hub portion;
wherein the catheter is encouraged to bow downward within the closed slotted passageway, from the top portion down toward the bottom portion.

14. The fluid transfer device of claim 12, wherein the slotted passageway comprises a closed slotted passageway including:
a top portion generally aligned with the distal opening; and
a bottom portion vertically below the top portion, with the bottom portion ending at a location vertically between the center axis and the bottom surface of the hub portion;
wherein the catheter is encouraged to bow downward within the closed slotted passageway, from the top portion down toward the bottom portion.

15. The fluid transfer device of claim 13, wherein the proximal opening, the distal opening, and the top portion are aligned with a tubing path along which the length of the catheter extends between the actuator and the distal end portion of the introducer.

16. The fluid transfer device of claim 13, wherein the proximal opening, the distal opening, and the top portion are offset vertically downward from a tubing path along which the length of the catheter extends between the actuator and the distal end portion of the introducer, thereby preloading the catheter to force a downward bowing thereof.

17. The fluid transfer device of claim 12, wherein a proximal end of the catheter support is configured to engage the actuator, such that during movement of the actuator to move the catheter from the first position to the second position, the actuator contacts the proximal end to move the catheter support with respect to the introducer.

18. A fluid transfer device for advancing a catheter into an indwelling catheter of a vascular access device, the fluid transfer device comprising:
an introducer having a proximal end portion and a distal end portion, the distal end portion coupleable to the vascular access device, the introducer defining an inner volume configured to movably receive the catheter;
an actuator movably coupled to the introducer and configured to move relative to the introducer to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter; and
a catheter support movably coupled to the introducer, and positioned between the actuator and the distal end portion of the introducer, the catheter support comprising a bracket portion and a hub portion, the hub portion defining a passageway therein that receives the catheter to provide support to a length of the catheter that extends between the actuator and the distal end portion of the introducer;
wherein the passageway comprises a non-linear passageway configured to pre-bias a downward bowing of the catheter as the catheter enters and exits the catheter support.

19. The fluid transfer device of claim 18, wherein the non-linear passageway comprises an arcuate passageway, with a center region of the arcuate passageway between the proximal end and the distal end of the hub portion being offset vertically upward from a proximal opening and a distal opening of the passageway, the curved passageway pre-biasing a downward bowing of the catheter.

20. The fluid transfer device of claim 18, wherein the non-linear passageway comprises sinusoidal passageway that shifts vertically as it runs between the proximal end and the distal end of the hub portion, the sinusoidal passageway pre-biasing a downward bowing of the catheter.
